(19) United States
(12) Patent Application Publication
(10) Pub. No.: US 2025/0040797 A1
(43) Pub. Date: Feb. 6, 2025

(54) LENS WASH AND IRRIGATION FOR ENDOSCOPY

(71) Applicant: Boston Scientific Scimed, Inc., Maple Grove, MN (US)

(72) Inventors: Ryan Evers, Billerica, MA (US); Ryan V. Wales, Northborough, MA (US); Collin Murray, Bolton, MA (US)

(73) Assignee: Boston Scientific Scimed, Inc., Maple Grove, MN (US)

(21) Appl. No.: 18/787,037

(22) Filed: Jul. 29, 2024

Related U.S. Application Data
(60) Provisional application No. 63/517,533, filed on Aug. 3, 2023.

(51) Int. Cl. A61B 1/12; A61B 1/00; A61B 1/005; A61B 90/70

(52) U.S. Cl. CPC A61B 1/126; A61B 1/00068; A61B 1/005; A61B 90/70; A61B 2090/701

(57) ABSTRACT

Devices, systems, and methods for a fluid supply for an endoscope. An expandable gas chamber presses against a saline chamber, allowing for the supply of pressurized saline to both a lens wash line and an irrigation line. As the saline chamber is emptied and decreases in volume the gas chamber receives more gas and increases in volume to maintain the fluid pressure.

### CROSS-REFERENCE TO RELATED APPLICATIONS

This application claims the benefit of U.S. Provisional Patent Application Ser. No. 63/517,533 filed on Aug. 3, 2023, the disclosure of which is incorporated herein by reference.

### TECHNICAL FIELD

This disclosure relates generally to valve assemblies and methods, and particularly to liquid supply during endoscopy

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, multi-chamber fluid container for a medical device, can include a fluid chamber in fluid communication with a first fluid outlet and a second fluid outlet and a gas chamber in mechanical contact with the fluid chamber, the gas chamber receiving gas to apply pressure to the fluid chamber in order to maintain fluid pressure within the fluid chamber.

Alternatively or additionally to any of the examples above, the fluid chamber can be a bag comprising a flexible material such that the bag decreases in volume as fluid flows out of the first and second fluid outlets.

Alternatively or additionally to any of the examples above, the gas chamber can be a bag comprising a flexible material such that the bag increases in volume as the gas chamber receives gas to apply pressure to the fluid chamber.

Alternatively or additionally to any of the examples above, the gas chamber can further include a pole mount suitable for hanging the container on a conventional pole for an intravenous fluid supply bag.

Alternatively or additionally to any of the examples above, the gas chamber can be in fluid communication with a gas cartridge.

Alternatively or additionally to any of the examples above, the fluid container further includes a rigid body containing the fluid chamber and the gas chamber, the rigid body defining a volume that does not expand or contract in response to fluid flow into or from the chamber.

Alternatively or additionally to any of the examples above, the rigid body can include an openable portion for replacement of the saline chamber.

Alternatively or additionally to any of the examples above, the fluid container can further include a saline source in fluid communication with the saline chamber.

Alternatively or additionally to any of the examples above, the saline source can be an intravenous saline supply bag configured to supply a saline drip to the saline chamber.

Alternatively or additionally to any of the examples above, the fluid container can further include an air pump in fluid communication with the gas chamber.

Alternatively or additionally to any of the examples above, the air pump can be a user-actuated bellows pump.

Alternatively or additionally to any of the examples above, the user-actuated bellows pump can have a greater air capacity than the gas chamber.

Alternatively or additionally to any of the examples above, the fluid chamber can contain saline.

In another example, an endoscopic surgical device an endoscopic probe having a lens wash feed line and an irrigation feed line and a fluid container according to any of the examples above, wherein the first fluid outlet supplies fluid to the lens wash feed line and the second fluid outlet supplies fluid to the irrigation feed line.

Alternatively or additionally to any of the examples above, the device can further comprise a first valve actuated by a user to open the lens wash feed line, and a second valve actuated by a user to open the irrigation feed line.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a cross-section side view of an illustrative multi-chamber bag.

FIG. 4 depicts a cross-section side view of an illustrative multi-chamber bag.

FIG. 4A depicts a cross-section side view of the bag from FIG. 4 along the A-A section line.

FIG. 5 depicts a cross-section side-view of an illustrative multi-chamber container with a rigid body.

FIG. 6 depicts a cross-section side-view of an illustrative multi-chamber container with a rigid body.

FIG. 7 depicts a schematic side-view of an illustrative system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a” “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

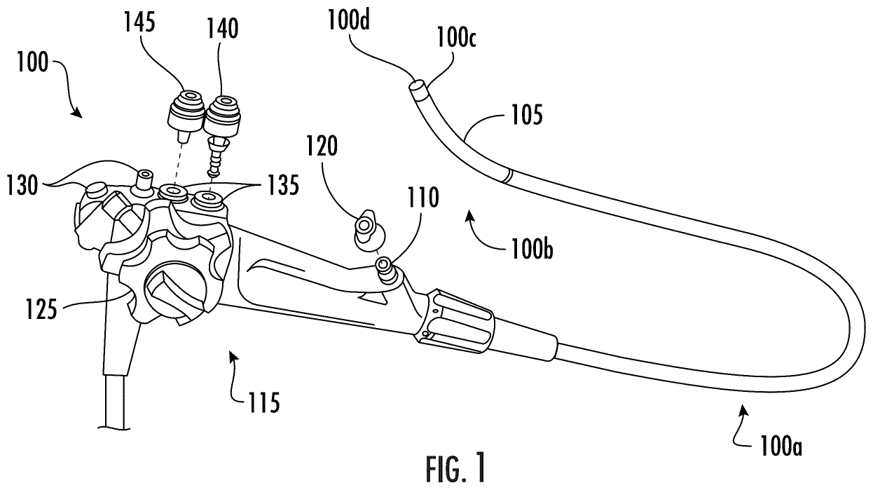

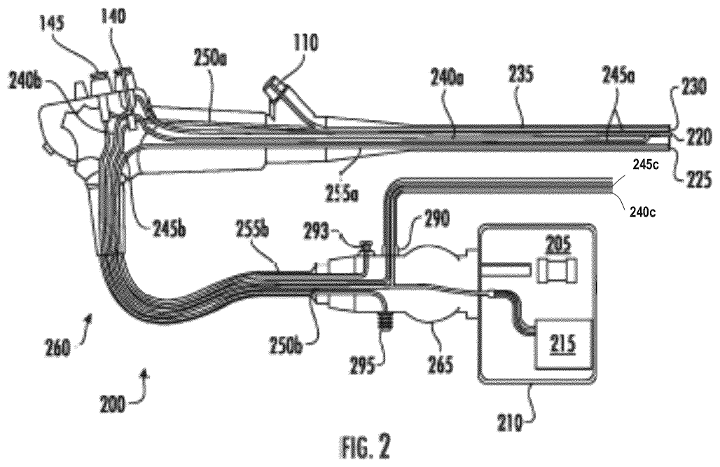

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serve as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir is fluidly connected to the endoscope 100 through the connector portion 265 and the umbilical 260. The gas feed line 240b from the umbilical 260 branches in the connector portion 265 to fluidly communicate with a length of gas supply tubing 240c at the detachable gas/lens wash connection 290, as well as the air pump 215. A length of lens wash tubing 245c runs to the same detachable connection 290 as the gas supply tubing 240c on the connector portion 265. In other embodiments, the connections may be separate and/or separated from each other. The connector portion 265 may also have a detachable irrigation connection 293 for irrigation supply tubing running from a source of irrigation water to the irrigation feed line 255b in the umbilical 260. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

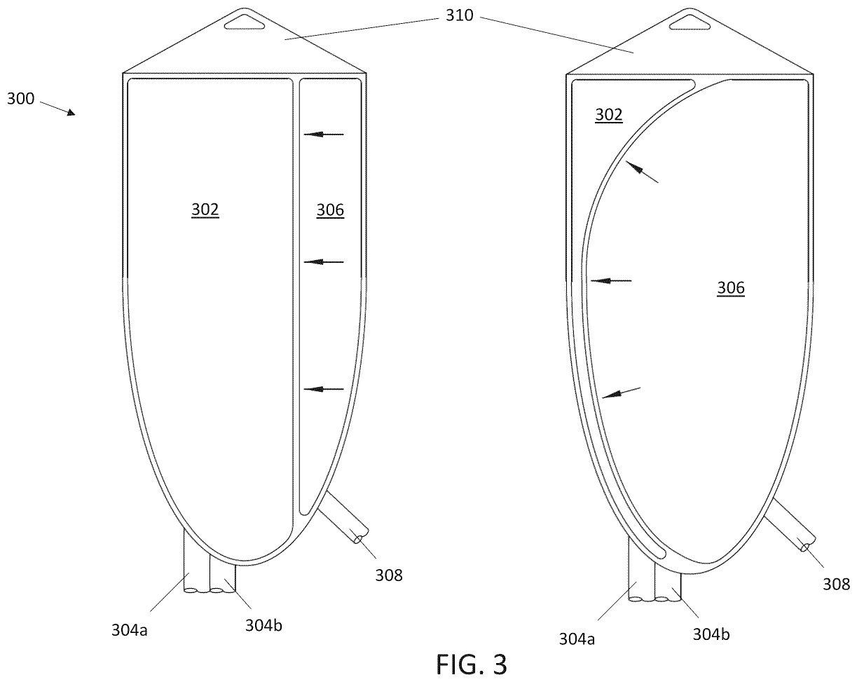

Both the lens wash feed line and the irrigation feed line may be fed from a multi-chamber bag 300 as illustrated in FIG. 3. A sealed saline bladder 302 acts as the water source for both feed lines. Saline outlet tubes 304a and 304b are attached to the saline bladder 302 and may be affixed by means of a known fluid interface or may be integrally formed with the bladder 302. Saline outlet tube 304a connects with the lens wash feed line 245b by any known tubing interface (not shown), which may also include a check valve or other mechanism to assure that fluid from the feed line 245b does not back up into the saline bag 302. As the multi-chamber bag 300 may be shaped and configured similar to that of an intravenous (IV) supply bag, an IV access spike or other compatible connector may be used.

Similarly, saline outlet tube 304b interfaces and provides fluid to the irrigation feed line 255b, which again may include one or more tubing elements to assure that the feed is one-way.

A pressurized gas bladder 306 presses against the saline bladder 302, maintaining the pressure needed for both lens wash and irrigation functions. A gas inlet tube 308 provides additional gas as needed to the gas bladder 306 to maintain the pressure on the saline bladder 302. Any source of gas may be attached to the gas inlet tube 308, such as a pressurized CO2 cartridge or a pump for room air. In some implementations, an exchangeable tubing interface may be included for quick replacement of the multi-chamber bag 300 as needed.

The multi-chamber bag 300 may include a pole mount 310 suitable for hanging on a conventional pole used for IV supply bags, on a wall hook, or on a fixture provided with other components of the endoscopic system.

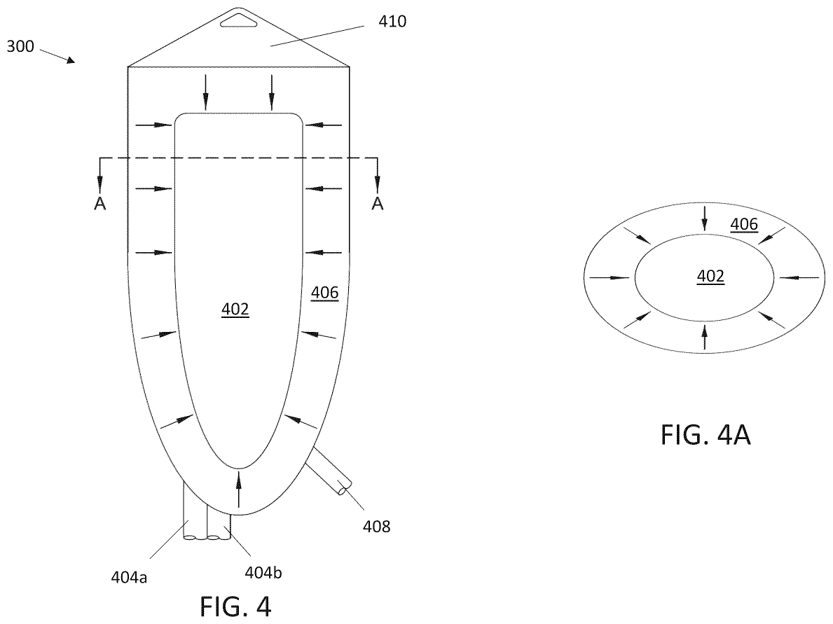

FIGS. 4 and 4A show an alternative embodiment of the multi-chamber bag 400 in which the saline bladder 402 is surrounded by the gas chamber 406 on all sides. Although shown as rotationally symmetrical, in some implementations of the multi-chamber bag 400, some portions of the gas chamber 406 may be thicker than others. The multi-chamber bag is attached to the gas inlet tube 408, lens wash outlet tube 404a, and irrigation outlet tube 404b as described above, and a pole mount 410 again allows the chamber to be hung on any suitable fixture.

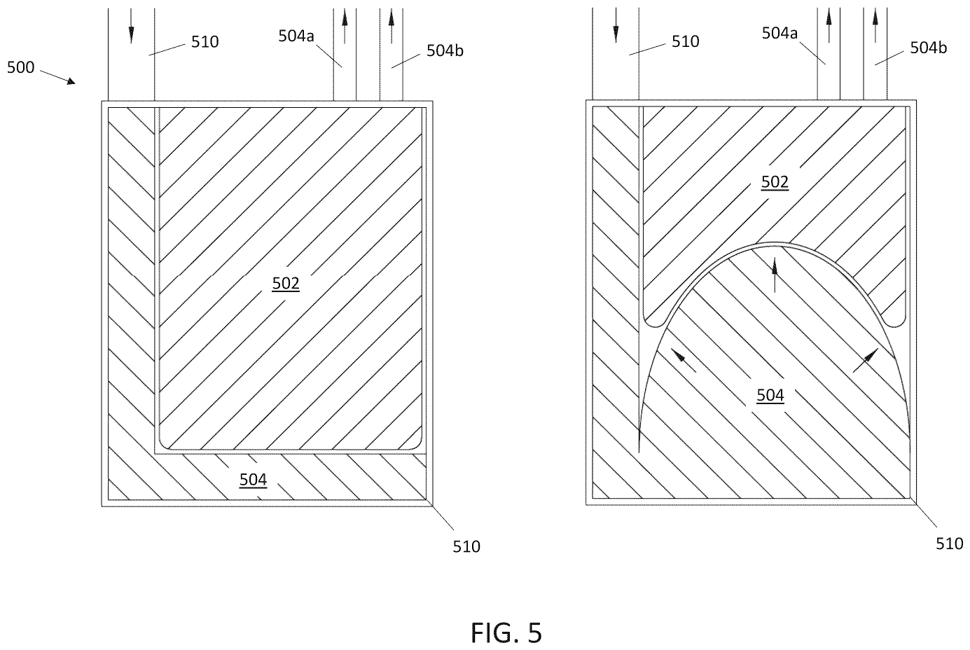

FIG. 5 shows a multi-chamber vessel 500 in which the saline bladder 502 and gas bladder 506 are surrounded by a rigid body 510. The body 510 may be of any appropriate material such as hard plastic or metal. Pressurized saline solution is provided from the saline bladder 502 to each of the lens wash outlet tube 504a and irrigation feed line 504b, while gas is supplied through the gas feed line 508 to the gas bladder 504 ensured sufficient pressure is applied against the saline bladder 502. In some implementations, the shape of the gas bladder 504 may be selected for the size and shape of the rigid body 510 in which it will set.

In some implementations, the multi-chamber vessel 500 may be single-use, to be disposed of when the saline bladder 502 is emptied or a particular procedure is completed. The rigid body 510 may then be permanently fastened or sealed, packaged, and shipped with the saline supply inside.

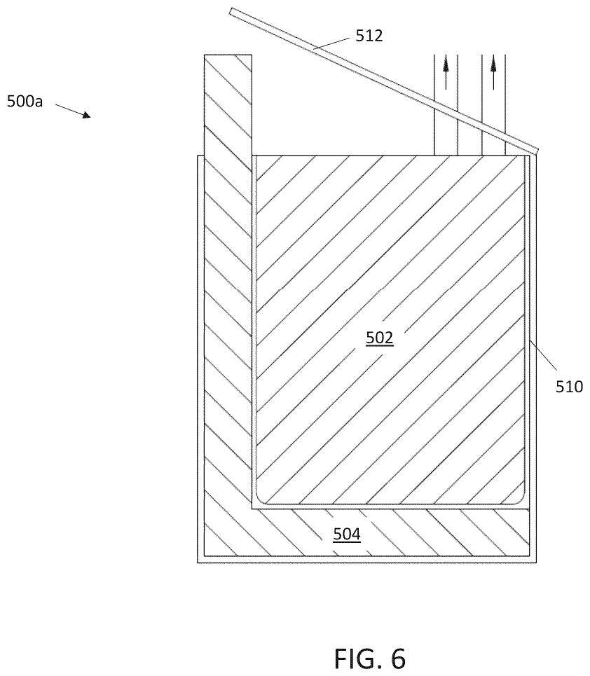

In other implementations, the multi-chamber vessel 500 may be designed for repeated use, such as when the rigid body 502 is made of a more durable material such as metal. FIG. 6 illustrates a multi-chamber vessel 500a with all components as described above with respect to the multi-chamber vessel 500, but with a top portion 512 of the rigid body 510 being openable for replacement of the saline bladder 502 therein. The gas bladder 504 may also be removable, or the gas bladder 504 may be permanently or semi-permanently affixed to the rigid body 510 and not intended to be replaced when the saline bladder 502 is replaced.

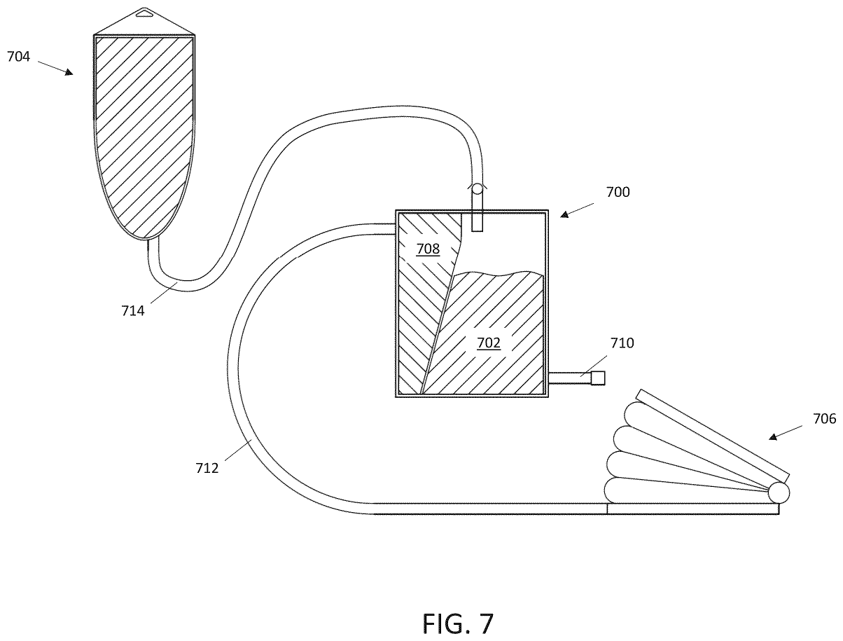

FIG. 7 illustrates another implementation using a drip chamber in conjunction with a user-actuated pump. The drip chamber 700 includes a saline chamber 702 fed by a saline source 704. The saline source 704 may be a conventional IV saline bag and may be fed into the saline chamber 702 by means of an IV spike and conventional IV drip line.

A foot-actuated bellows pump 706 forces air into a gas chamber 708, which forces pressurized saline out of the saline chamber 702 by means of an outlet 710. As described above, the outlet 710 may include tubing for both a lens wash feed line and an irrigation feed line. When the pump 706 is released, air is forced back out of the gas chamber 708. This may be due to the elastic material of the chamber 708, by a spring mechanism included in the device, or by suction coming from opening of the pump 706.

In some implementations, the pump 706 may be configured to move a greater volume of air in a single actuation than the volume of the chamber 700, in order force all or substantially all of the saline from the drip chamber 700 on each actuation of the pump 706. Depending on the mechanism, check valves may be included in the gas inlet tube 712 between the pump 706 and gas chamber 708, in the saline inlet tube 714 between the saline source 704 and the saline chamber 702, and in each tube within outlet 710. Saline from the source 702 refills the saline chamber 704 each time it is used.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A multi-chamber fluid container for a medical device, comprising:
a fluid chamber in fluid communication with a first fluid outlet and a second fluid outlet; and
a gas chamber in mechanical contact with the pressurized fluid chamber, the gas chamber receiving gas to apply pressure to the fluid chamber in order to maintain fluid pressure within the fluid chamber.

2. The fluid container of claim 1, wherein the fluid chamber is a bag comprising a flexible material such that the bag decreases in volume as fluid flows out of the first and second fluid outlets.

3. The fluid container of claim 1, wherein the gas chamber is a bag comprising a flexible material such that the bag increases in volume as the gas chamber receives gas to apply pressure to the fluid chamber.

4. The fluid container of claim 1, comprising a pole mount suitable for hanging the container on a conventional pole for an intravenous fluid supply bag.

5. The fluid container of claim 1, wherein the gas chamber is in fluid communication with a gas cartridge.

6. The fluid container of claim 1, further comprising a rigid body containing the fluid chamber and the gas chamber, the rigid body defining a volume that does not expand or contract in response to fluid flow into or from the chamber.

7. The fluid container of claim 6, wherein the rigid body includes an openable portion for replacement of the saline chamber.

8. The fluid container of claim 1, further comprising a saline source in fluid communication with the saline chamber.

9. The fluid container of claim 8, wherein the saline source is an intravenous saline supply bag configured to supply a saline drip to the saline chamber.

10. The fluid container of claim 1, further comprising an air pump in fluid communication with the gas chamber.

11. The fluid container of claim 10, wherein the air pump is a user-actuated bellows pump.

12. The fluid container of claim 11, wherein the user-actuated bellows pump has a greater air capacity than the gas chamber.

13. The fluid container of claim 1, wherein the fluid chamber contains saline.

14. An endoscopic surgical device, comprising:
an endoscopic probe having a lens wash feed line and an irrigation feed line; and
a fluid container, comprising:
a fluid chamber in fluid communication with a first fluid outlet supplying fluid to the lens wash feed line and a second fluid outlet supplying fluid to the fluid container; and
a gas chamber in mechanical contact with the fluid chamber, the gas chamber receiving gas to apply pressure to the fluid chamber in order to maintain fluid pressure within the fluid chamber.

15. The device of claim 14, further comprising a first valve actuated by a user to open the lens wash feed line, and a second valve actuated by a user to open the irrigation feed line.

16. The device of claim 14, wherein the fluid chamber is a bag comprising a flexible material such that the bag decreases in volume as fluid flows out of the first and second fluid outlets.

17. The device of claim 14, wherein the gas chamber is a bag comprising a flexible material such that the bag increases in volume as the gas chamber receives gas to apply pressure to the fluid chamber.

18. The device of claim 14, wherein the gas chamber is in fluid communication with a gas cartridge.

19. The device of claim 14, wherein the fluid container further comprises a rigid body containing the fluid chamber and the gas chamber, the rigid body defining a volume that does not expand or contract in response to fluid flow into or from the chamber.

20. The device of claim 14, further comprising an air pump in fluid communication with the gas chamber.
